(19) United States
(12) Patent Application Publication
(10) Pub. No.: US 2013/0197339 A1
(43) Pub. Date: Aug. 1, 2013

(54) METHOD OF MONITORING DEPTH OF ANESTHESIA AND APPARATUS FOR SAME

(71) Applicant: NEUROCHIP CORPORATION, Toronto (CA)

(72) Inventors: Berj L. BARDAKJIAN, Toronto (CA); Eunji Ellen KANG, Richmond Hill (CA)

(73) Assignee: NEUROCHIP CORPORATION, Toronto (CA)

(21) Appl. No.: 13/755,634

(22) Filed: Jan. 31, 2013

Related U.S. Application Data
(60) Provisional application No. 61/593,188, filed on Jan. 31, 2012.

(51) Int. Cl. A61B 5/00; A61B 5/0478; A61B 5/04

(52) U.S. Cl. CPC A61B 5/4821; A61B 5/04012; A61B 5/4839; A61B 5/0478; A61B 5/742
USPC 600/383; 600/544

(57) ABSTRACT

A method of monitoring depth of anesthesia comprises generating differential EEG signals from EEG signals acquired by electrode pairs during an anesthesia protocol, processing the differential EEG signals to determine at least one signal feature and monitoring changes in the at least one signal feature to determine depth of anesthesia. The at least one signal feature comprises at least one of normalized power of the differential EEG signals acquired from at least one of the electrode pairs, and wavelet bicoherence between the differential EEG signals acquired from at least two of the electrode pairs.

### CROSS-REFERENCE TO RELATED APPLICATIONS

This application claims the benefit of U.S. Provisional Application No. 61/593,188 filed on Jan. 31, 2012 entitled “METHOD OF MONITORING DEPTH OF ANESTHESIA AND APPARATUS FOR SAME”, the entire disclosure of which is incorporated herein by reference.

### FIELD OF THE INVENTION

The present invention relates generally to anesthesia and in particular, to a method of monitoring depth of anesthesia and an apparatus for same.

### BACKGROUND OF THE INVENTION

Anesthesia is typically used during surgery to maintain patients at a level of unconsciousness that is deep enough to enable them to undergo the surgery without pain or recall. It is undesirable to administer an excessive amount of anesthesia, as doing so can prolong the post-anesthesia recovery period following surgery. Determining the proper amount of anesthesia to administer requires a reliable measure of the depth of anesthesia (DoA) of the patient during the surgery.

One approach to measuring DoA involves monitoring electroencephalographic (EEG) signals acquired from the patient during the administration of anesthesia. Several methods for monitoring EEG signals to determine DoA have been described. For example, one method involves use of a Bispectral Index (BIS™) monitor, developed by Aspect Medical Inc., Newton, Mass., U.S.A., which utilizes a proprietary algorithm for processing EEG signals to measure DoA.

Other methods have also been described. For example, U.S. Pat. No. 6,934,579 to Mantzaridis et al. discloses an anesthesia control system and a method of calculating an index representative of DoA. The method comprises subjecting a patient to a repetitive audio stimulus and monitoring auditory evoked potentials (AEP) produced by the patient, and then recording these auditory evoked potentials using EEG recording means, and providing a signal corresponding to the coarseness of the monitored AEP signal and using this signal as an index indicative of anesthetic depth. The raw AEP signal is divided into a series of sweeps. Each sweep is synchronized with the repetitive audio stimulus, and sweeps are recorded in sequence to produce a time averaged sweep from which the anesthetic index is calculated. The anesthetic index is constantly updated by repeatedly conducting a successive series of sweeps. The system and index signal can be used as part of an anesthesia control system for regulating the supply of anesthetic to the patient to maintain the anesthetic index at a predetermined level.

U.S. Pat. No. 7,805,187 to Sarkela et al. discloses a method and apparatus for monitoring the anesthetic state of a subject. Physiological signal data including EEG and possibly also EMG signal components is acquired from a subject and supplied to a monitoring process configured to derive, based on the data, a state index indicative of the anesthetic state of the subject. In order to extend the applicability of conventional monitoring processes, the operation of the monitoring process is controlled according to whether at least one drug inducing high-frequency EEG signal components is administered to the subject. The presence of the high-frequency EEG signal components in the physiological signal data may be detected automatically.

It may be challenging using some known EEG-based DoA monitoring methods to identify and measure changes in the different levels of consciousness following loss of consciousness (LoC), especially during the maintenance phase of the anesthesia administration protocol. Improvements are thus desired. It is therefore an object of the invention to provide a novel method of monitoring depth of anesthesia and an apparatus for the same.

### SUMMARY OF THE INVENTION

Accordingly, in one aspect, there is provided a method of monitoring depth of anesthesia comprising: generating differential EEG signals from EEG signals acquired by electrode pairs during an anesthesia protocol; processing the differential EEG signals to determine at least one signal feature; and monitoring changes in the at least one signal feature to determine depth of anesthesia, wherein the at least one signal feature at least one of normalized power of the differential EEG signals acquired from at least one of the electrode pairs, and wavelet bicoherence between the differential EEG signals acquired from at least two of the electrode pairs.

In one embodiment, the wavelet bicoherence is calculated between 5 to 18 Hz and 10 to 40 Hz frequency ranges of the differential EEG signals. The normalized power may be calculated within a 4 to 8 Hz frequency range of the differential EEG signals. The method may further comprise calculating a required dosage value of anesthesia based on the monitoring and delivering a controllable quantity of anesthesia to a patient based on the calculated required dosage value.

In another aspect there is provided a system for monitoring depth of anesthesia comprising: electrodes configured to be placed on a subject's scalp; and processing structure for generating differential EEG signals from EEG signals acquired by electrode pairs during an anesthesia protocol, the processing structure being configured to: process the differential EEG signals to determine at least one signal feature, and determine changes in the at least one signal feature during the anesthesia protocol, wherein the at least one signal feature comprises at least one of normalized power of the differential EEG signals acquired from at least one of the electrode pairs, and wavelet bicoherence between the differential EEG signals acquired from at least two of the electrode pairs.

In still another aspect there is provided a computer-readable medium having embodied thereon a computer program for monitoring depth of anesthesia, the computer program comprising instructions which, when executed by processing structure, cause an apparatus at least to: process differential EEG signals generated from EEG signals acquired by electrode pairs during an anesthesia protocol to determine at least one signal feature; and monitor changes in the at least one signal feature to determine depth of anesthesia, wherein the at least one signal feature comprises at least one of normalized power of the differential EEG signals acquired from at least one of the electrode pairs, and wavelet bicoherence between the differential EEG signals acquired from at least two of the electrode pairs.

In still yet another aspect, there is provided an apparatus comprising: processing structure; and memory in communication with the processing structure, the memory storing computer-readable code, the computer-readable code when executed by the processing structure causing the apparatus at least to: process differential EEG signals generated from EEG signals acquired by electrode pairs during an anesthesia protocol to determine at least one signal feature; and monitor changes in the at least one signal feature to determine depth of anesthesia, wherein the at least one signal feature comprises at least one of normalized power of the differential EEG signals acquired from at least one of the electrode pairs, and wavelet bicoherence between the differential EEG signals acquired from at least two of the electrode pairs.

### BRIEF DESCRIPTION OF THE DRAWINGS

Embodiments will now be described more fully with reference to the accompanying drawings in which:

FIG. 1 is a schematic diagram of an apparatus for monitoring depth of anesthesia;

FIG. 2 is a schematic diagram of an another embodiment of an apparatus for monitoring depth of anesthesia;

FIG. 3 is a schematic view of an electrode arrangement for acquiring EEG signals;

FIG. 4 is a graphical plot of squared-amplitude, band-limited signals, calculated for differential EEG signals acquired during an anesthesia protocol from the F4-Fz electrode pair, for frequency band ranges 0 to 4 Hz and 12 to 16 Hz;

FIG. 5 is a graphical plot of average normalized power of differential EEG signals acquired during the anesthesia protocol from frontal and posterior electrode pairs;

FIG. 6 is a graphical plot of relative phase coherence between differential EEG signals acquired during the anesthesia protocol from bilateral sets of electrode pairs (F3-F7 and F8-F4) and (T5-P3 and T6-P4), and from frontal-posterior sets of electrode pairs (F8-F4 and T4-C4) and (T4-C4 and T6-P4);

FIG. 7A is a graphical plot of relative phase coherence between differential EEG signals acquired during the anesthesia protocol from a bilateral set of electrode pairs (F3-F7 and F8-F4) and from a frontal-posterior set of electrode pairs (F8-F4 and T4-C4);

FIG. 7B is a graphical plot of relative phase coherence between differential EEG signals acquired during the anesthesia protocol from a frontal-posterior set of electrode pairs (F8-F4 and 14-C4), for frequency band ranges 8 to 14 Hz and 40 to 100 Hz;

FIG. 8A is a graphical plot of wavelet bicoherence between differential EEG signals acquired during the anesthesia protocol from a bilateral set of electrode pairs (F3-F7 and F8-F4);

FIG. 8B is a graphical plot of mean wavelet bicoherence between differential EEG signals acquired from six patients during the anesthesia protocol from a bilateral set of electrode pairs (F3-F7 and F8-F4) for frequencies of 5 to 18 Hz and 10 to 40 Hz;

FIG. 9A is a graphical plot of modulation index of differential EEG signals acquired during the anesthesia protocol;

FIG. 9B is a graphical plot of mean modulation index of differential EEG signals acquired from the six patients during the anesthesia protocol, calculated for a 15 to 30 Hz phase and a 60 to 200 Hz amplitude; and

FIG. 10 is a graphical plot of mean Bispectral Index of differential EEG signals acquired from the six patients during the anesthesia protocol.

### DETAILED DESCRIPTION OF THE EMBODIMENTS

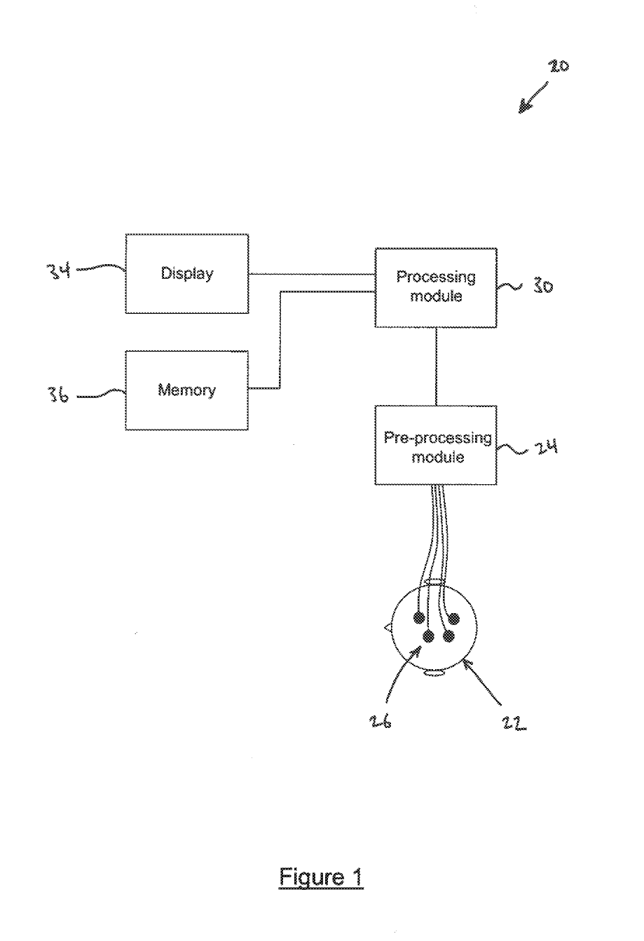

Turning now to FIG. 1, an apparatus for monitoring depth of anesthesia is shown and is generally indicated by reference numeral 20. Apparatus 20 is configured to process electroencephalographic (EEG) signals acquired during an anesthesia protocol to which a patient 22 is subjected, so as to determine one or more signal features of the EEG signals. Changes in the signal features are monitored during the anesthesia protocol to determine depth of anesthesia (DoA) of the patient 22.

Apparatus 20 comprises a pre-processing module 24 that is in communication with a plurality of electrodes 26 positioned on the scalp of the patient 22. The pre-processing module 24 is configured to receive incoming analog EEG signals from the electrodes 26, to amplify the analog EEG signals as necessary, and to convert the amplified analog EEG signals to digital EEG signals for output.

Apparatus 20 also comprises a processing module 30 that is in communication with the pre-processing module 24. The processing module 30 is configured to receive the digital EEG signals output by the pre-processing module 24, and to calculate differences between two (2) digital EEG signals, each obtained from a respective electrode 26, so as to yield differential EEG signals. The processing module 30 is also configured to carry out signal processing of the differential EEG signals to calculate the one or more signal features.

Apparatus 20 also comprises a display 34 and a memory 36 that are in communication with the processing module 30. The processing module 30 is configured to output the differential EEG signals and the calculated signal features to the display 34, where they may be visually monitored in real time by an operator, such as for example an anesthesiologist, during the anesthesia protocol. The processing module 30 is also configured to output the digital EEG signals, the differential EEG signals, and the calculated signal features to the memory 36 for storage. The stored digital EEG signals, differential EEG signals, and calculated signal features may then be later retrieved and processed, as desired.

In this embodiment, the pre-processing module 24, the processing module 30, the display 34 and the memory 36 are embodied within a single general purpose computing device, namely a laptop, tablet or personal computer or other suitable processing structure.

The signal features calculated by the processing module 30 in this embodiment comprise (i) normalized power, (ii) relative phase coherence, (iii) wavelet bicoherence, and (iv) modulation index. The methods used to calculate each of these signal features are described below.

Normalized power is determined by first decomposing each differential EEG signal into a plurality of narrow band-limited signals using a finite impulse response filter of order N. In this embodiment, the value of order N is 5000, and the frequency band ranges into which each differential EEG signal is decomposed are 0 to 4 Hz, 4 to 8 Hz, 8 to 12 Hz, 12 to 16 Hz, 16 to 20 Hz, 20 to 30 Hz, 30 to 50 Hz and 50 to 80 Hz.

The amplitude of each band-limited signal is then squared, and values of squared amplitude are summed over a predefined time period, t, to yield a signal power value, P_{S}. The signal power value P_{S} is then divided by a baseline power value, P_{B}, for the corresponding frequency band to yield a normalized power value, P_{N}, described by:

$${P}_{N} = \frac{{P}_{S}}{{P}_{B}} \qquad ( 1 )$$

The baseline power value, P_{B}, is obtained from a baseline differential EEG signal that is acquired during an initial “baseline” period prior to administration of any anesthesia, and which is decomposed using the finite impulse response filter. The amplitude of each band-limited baseline signal is squared, and values of squared amplitude are then summed over a predefined baseline time period, t_{B}, to yield the baseline power value P_{B}.

In this embodiment, the processing module 30 calculates an instantaneous value of normalized power P_{N}ⁱ using an instantaneous value of signal power value P_{S}ⁱ, which is obtained in a dynamic manner for time windows of t=30 seconds, as described by:

$${P}_{N}^{i} = \frac{{P}_{S}^{i}}{{P}_{B}} \qquad ( 2 )$$

where, for this embodiment, the values of t and t_{B} are 30 seconds.

Relative phase coherence is determined by first calculating a continuous wavelet transform of a signal, x(t), to yield a complex-valued wavelet coefficient matrix, described by:

W(σ,τ)=w(σ,τ)+i\tilde{w}(σ,τ) (3)

where w and \tilde{w} are the real and complex wavelet coefficients, respectively, over a scale σ and with a time shift τ.

Relative phase difference, Δφ(t), between two general signals, y₁(t) and y₂(t), is generally described by:

$$Δ φ ( t ) = arctan \frac{{\tilde{y}}_{1} ( t ) {y}_{2} ( t ) - {y}_{1} ( t ) {\tilde{y}}_{2} ( t )}{{y}_{1} ( t ) {y}_{2} ( t ) + {\tilde{y}}_{1} ( t ) {\tilde{y}}_{2} ( t )} . \qquad ( 4 )$$

Accordingly, the relative phase difference between complex wavelet coefficient matrices derived from two signals, namely W₁(σ,τ) and W₂(σ,τ), is described by:

$$Δ φ ( σ , τ ) = arctan \frac{{\tilde{w}}_{1} ( σ , τ ) {w}_{2} ( σ , τ ) - {w}_{1} ( σ , τ ) {\tilde{w}}_{2} ( σ , τ )}{{w}_{1} ( σ , τ ) {w}_{2} ( σ , τ ) + {\tilde{w}}_{1} ( σ , τ ) {\tilde{w}}_{2} ( σ , τ )} . \qquad ( 5 )$$

The relative phase coherence, ρ, between two signals for a given scale (˜1/frequency) and a given time segment, T=[τ−Δτ,τ+Δτ], is described by:

ρ(σ,T)= [symbol] (exp(iΔφ(σ,T)) [symbol] (6)

where the angled brackets denote a time-average, and where the value of the relative phase coherence, ρ, ranges from 0 to 1.

In this embodiment, the processing module 30 calculates the continuous wavelet transform for each of two (2) differential EEG signals according to Equation (3), and then calculates values of the relative phase coherence for the continuous wavelet transforms according to Equation (6).

As will be appreciated, wavelet bicoherence generally represents the degree of higher-order coupling between different frequency bands. The XYX wavelet cross-spectrum is described by:

B_{xyz}(σ₁,σ₂,T)=∫_{T}W_{X}(σ₁,τ)W_{Y}(σ₂,τ)W*_{X}(σ,τ)dτ (7)

where 1/σ=1/σ/σ₁+1/σ₂, and where the asterisk denotes the complex conjugate. The XYX wavelet bicoherence is therefore the cross-bispectnim normalized in power to the range 0 to 1, as described by:

$${b}_{xyx} ( {σ}_{1} , {σ}_{2} , T ) = \frac{\vert {B}_{xyx} ( {σ}_{1} , {σ}_{2} , T ) \vert}{\sqrt{{\int }_{T} {\vert {W}_{X} ( {σ}_{1} , τ ) {W}_{Y} ( {σ}_{2} , τ ) \vert}^{2} d τ {\int }_{T} {\vert {W}_{X} ( σ , τ ) \vert}^{2} d τ}} . \qquad ( 8 )$$

Modulation index generally represents phase-to-amplitude coupling of two (2) differential EEG signals. The modulation index is calculated in accordance with an approach described in the publication by Canolty et al., entitled “High gamma power is phase-locked to theta oscillations in human neocortex,” Science, vol. 313, pp. 1626 to 1628, (2006). Each differential EEG signal is first decomposed into a plurality of narrow band-limited signals using a finite impulse response filter of order N=5000. Each band-limited signal is then standard-normalized by mean-subtraction, followed by division by the standard deviation, to a obtain a standard normalized band-limited signal, x_{f}(t). The Hilbert Transform is then applied to the standard normalized band-limited signal x_{f}(t) to yield a complex-valued analytic signal, z_{f}(t), described by:

z_{f}(t)=x_{f}(t)+iH[x_{f}(t) (9)

From the analytic signal, an analytic phase time series, φ_{f}(t), and an analytic amplitude time series, A_{f}(t), are calculated. The analytic phase time series φ_{f}(t) is calculated by extracting the phase of z_{f}(t) at each time point, while the analytic amplitude time series A_{f}(t) is determined by calculating the absolute value of z_{f}(t). In this embodiment, the phase time series φ_{f}(t) is calculated over a frequency range of 0 to 30 Hz, with 1 Hz increments, while the amplitude time series A_{f}(t) is calculated over a frequency range 0 to 200 Hz, with 5 Hz increments. A composite function z_{new}(t) is then constructed by combining the phase time series φ_{f}(t) of one frequency band with the amplitude time series A_{f}(t) of another, as described by:

z_{new}(t)=A_{f}_{ₖ}(t)e^{iσ}^{_{fj}}^{(t)} (10)

The mean of composite function z_{new}(t) provides a good indication of the phase-to-amplitude coupling between the two signals. However, to offset any dominant contribution of either the phase time series φ_{f}(t) or the amplitude time series A_{f}(t) alone, a normalization is carried out by creating a surrogate signal z_{SUR}(t,τ_{d}) through introduction of a time delay τ_{d} between two signals, as described by:

z_{SUR}(t,τ_{d})=A_{f}_{ₖ}(t+τ_{d})e^{iφ}^{_{fj}}^{(t)} (11)

Values of surrogate signal z_{SUR}(t,τ_{d}) are calculated for several values of time delay τ_{d}, and from these values of surrogate signal z_{SUR}(t,τ_{d}) a mean, μ, and a standard deviation, σₛ, are obtained, and are in turn used to calculate a normalized mean, M_{NORM}, described by:

M_{NORM}=(M−μ)/σₛ (12)

where M is the mean of the composite function z_{new}(t). The value of the normalized mean M_{NORM} is referred to herein as the modulation index.

In use, during an anesthesia protocol, the pre-processing module 24 continuously receives analog EEG signals from electrode pairs 26, and then amplifies the EEG signals and converts the amplified EEG signals to digital EEG signals. The processing module 30 receives the digital EEG signals output by the pre-processing module 24, calculates differential EEG signals, and stores the differential EEG signals in memory 36. The processing module 30 also dynamically calculates the values of normalized power, relative phase coherence, wavelet bicoherence, and modulation index, in the manners described above, and stores these values in memory 36. The processing module 30 also calculates changes in the signal features by comparing the dynamically calculated values of normalized power, relative phase coherence, wavelet bicoherence, and modulation index with previously dynamically calculated values of these signal features stored in memory 36. The processing module 30 displays the dynamically calculated values of the signal features and the calculated changes in the signal features on the display 34 in real time, where they may be monitored by the operator to assess the DoA of the patient 22. In this embodiment, changes in the value of normalized power in the 4 to 8 Hz frequency range, and changes in the value of wavelet bicoherence between the 5 to 18 Hz and 10 to 40 Hz frequency ranges, are used to indicate changes in DoA during administration of anesthesia.

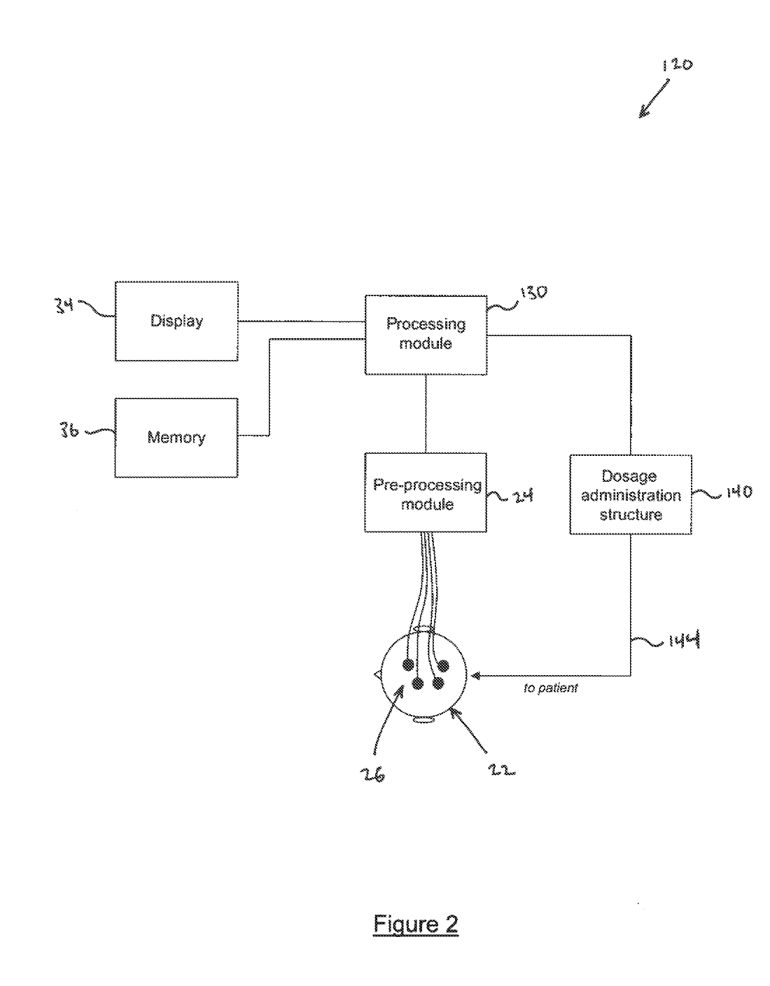

The apparatus 20 may also comprise structure for delivering a controlled dosage of anesthesia to a patient in accordance with measured depth of anesthesia. For example, FIG. 2 shows another embodiment of an apparatus for monitoring depth of anesthesia, and which is generally indicated by reference numeral 120. Similar to apparatus 20 described above and with reference to FIG. 1, apparatus 120 is configured to process EEG signals acquired during an anesthesia protocol to which a patient 22 is subjected, so as to determine one or more signal features of the EEG signals. The apparatus is also configured to monitor changes in the signal features during the anesthesia protocol to determine the DoA. Apparatus 120 is further configured to administer a controlled dosage of anesthesia to the patient in accordance with the signal features, so as to maintain the patient 22 at a desired DoA.

Similar to apparatus 20 described above, apparatus 120 comprises a pre-processing module 24 that is in communication with a plurality of electrodes 26 positioned on the scalp of the patient 22. The pre-processing module 24 is configured to receive incoming analog EEG signals from the electrodes 26, to amplify the analog EEG signals, and to convert the amplified analog EEG signals to digital EEG signals for output.

Apparatus 120 also comprises a processing module 130 that is in communication with the pre-processing module 24. The processing module 130 is configured to receive the digital EEG signals output by the pre-processing module 24, and to calculate differences between two (2) digital EEG signals, each obtained from a respective electrode 26, so as to yield differential EEG signals. The processing module 30 is also configured to carry out signal processing of the differential EEG signals to calculate the signal features.

Apparatus 120 also comprises a display 34 and a memory 36 that are in communication with the processing module 130. The processing module 130 is configured to output the differential EEG signals and the calculated signal features to the display 34, where they may be visually monitored in real time by an operator, such as for example an anesthesiologist, during the anesthesia protocol. The processing module 130 is also configured to output the digital EEG signals, the differential EEG signals and the calculated signal features to the memory 36 for storage. The stored digital EEG signals, differential EEG signals and calculated signal features may be later retrieved and processed, as desired.

In this embodiment, the pre-processing module 24, the processing module 130, the display 34 and the memory 36 are similarly embodied within a single general purpose computing device, namely a laptop, tablet or personal computer or other suitable processing structure.

The signal features calculated by the processing module 130 in this embodiment comprise (i) normalized power, (ii) relative phase coherence, (iii) wavelet bicoherence, and (iv) modulation index. These signal features are calculated in the same manners described above with reference to processing module 30.

The processing module 130 is also configured to compare the values of the signal features with previously calculated values of the signal features stored in memory 36. The processing module 130 is configured to automatically calculate a required dosage value of anesthesia required by the patient 22, based on this comparing, and to display the calculated required dosage value on the display 34.

The apparatus 120 also comprises an input device (not shown) that is in communication with the processing module 130. The input device is configured to convey input, in the form of a desired dosage value entered by the operator, to the processing module 130. In this embodiment, the input device is a keyboard.

The processing module 130 is in communication with dosage administration structure 140. The dosage administration structure 140 is configured to deliver a controllable quantity of anesthesia to the patient 22 via a fluid delivery line 144, in accordance with the calculated required dosage value, or in accordance with the desired entered dosage value if one has been entered, received from the processing module 130. In this embodiment, the dosage administration structure 140 comprises at least one reservoir (not shown) containing a volume of a respective medical fluid, fluid delivery hardware (not shown) configured to discharge a controlled volume of at least one medical fluid from the at least one reservoir into the delivery line 144, and fluid delivery hardware processing structure (not shown) configured to control the fluid delivery hardware in accordance with instructions received from the processing module 130. In this embodiment, the at least one medical fluid contained in the at least one reservoir comprises at least one anesthetic fluid, and the delivery line 144 comprises at least one fluid conduit. It will be understood that each medical fluid may be a liquid, a gas, or a mixture thereof.

In use, during an anesthesia protocol, the pre-processing module 24 continuously receives analog EEG signals from electrode pairs 26, and then amplifies the EEG signals and converts the amplified EEG signals to digital EEG signals. The processing module 130 receives the digital EEG signals output by the pre-processing module 24, calculates differential EEG signals, and stores the calculated differential EEG signals in memory 36. The processing module 130 also dynamically calculates the values of normalized power, relative phase coherence, wavelet bicoherence, and modulation index, in the manners described above, and stores these values in memory 36. The processing module 130 also calculates changes in the signal features by comparing the dynamically calculated values of normalized power, relative phase coherence, wavelet bicoherence, and modulation index with previously dynamically calculated values of these signal features stored in memory 36. The processing structure displays the dynamically calculated values of the signal features and the calculated changes in the signal features on the display 34 in real time, where they may be monitored by the operator to assess the DoA of the patient 22. In this embodiment, changes in the value of normalized power in the 4 to 8 Hz frequency range, and changes in the value of wavelet bicoherence between the 5 to 18 Hz and 10 to 40 Hz frequency ranges, used to indicate changes in DoA during administration of anesthesia.

The processing module 130 also monitors the calculated changes in the signal features and automatically calculates a required dosage value of anesthesia required by the patient 22, and displays the calculated required dosage value on the display 34.

If the processing module 130 receives input from the operator in the form of a desired entered dosage value, then the processing module 130 communicates this desired entered dosage value to the dosage administration structure 140. Otherwise, if no desired entered dosage value is received by the processing module 130, then the processing module 130 communicates the calculated required dosage value to the dosage administration structure 140.

The dosage administration structure 140 in turn delivers a controllable quantity of anesthesia to the patient 22 via a fluid delivery line 144, in accordance with the calculated required dosage value, or in accordance with the desired entered dosage value if one has been entered, received from the processing module 130.

The following example illustrates various applications of the above-described systems and methods.

### EXAMPLE

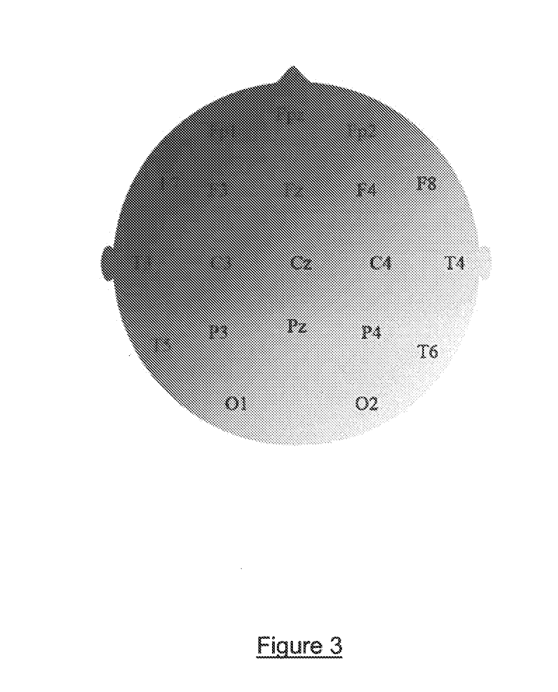

Digitized scalp EEG signals were sampled at 1024 Hz using disposable silver/silver-chloride electrodes, 9 mm in diameter (XLTEK, Oakville, Ontario, Canada). Electrode placement followed the international standard 10-20 system recommended by the International Federation of Societies for Electroencephalography and Clinical Neurophysiology (IFSECN), and as shown in FIG. 3. A common reference electrode was positioned between Cz and Fz. EEG signals were recorded from six (6) patients undergoing surgical procedures requiring general anesthesia and having a duration of approximately 1 hour. The patients were screened such that they were not being prescribed central nervous system (CNS) medications and did not have any diagnosed CNS pathology. EEG signals were recorded continuously just prior to and during anesthesia and surgery, and during the emergence period following anesthesia and surgery. The bispectral index (BIS™) was simultaneously recorded using a BIS™ monitor (Aspect, Mass., USA). Prior to data acquisition, approval from the University Health Network Research Ethics Board of Toronto, Canada, was obtained, and all patients provided written informed consent.

The anesthesia protocol referred to in this Example comprised three (3) periods, namely an initial “baseline” period prior to administration of any anesthesia, an “administration” period during which anesthesia was administered, and an “emergence” period following the “administration” period and during which no anesthesia was administered. The “administration” period comprised two phases, namely an induction phase in which inducing anesthetics were administered, and a subsequent maintenance phase in which maintenance anesthetics were administered.

General anesthesia was induced with fentanyl (2-3 μg/kg) and propofol (2.5 mg/kg), followed by continuous propofol infusion (140 μg/kg/min) to maintain an anesthetic plasma level of propofol at 6 μg/ml for 5 minutes to allow for EEG and BIS™ recordings at steady state. When the loss of the eyelash reflex was noted, patients were subsequently intubated. During induction of anesthesia, patients were routinely ventilated to keep arterial oxygen saturation (SaO₂) above 97%. During the maintenance phase of anesthesia, patients received a volatile anesthetic, sevoflurane, in an air/O₂ (1:1) mixture, to achieve a steady mean alveolar concentration (MAC) value of 1.2, and supplemental doses of fentanyl, as required. This was maintained for the duration of surgery.

Five (5) anesthetic states are refened to in this Example, namely “baseline”, “induction”, “1.0 MAC”, “steady MAC”, and “emergence”. The “baseline” state corresponds to a period of data collection from awake patients during the baseline period. The states of “induction,” “1.0 MAC” and “steady MAC” states occur within the “administration period”. Specifically, the “induction” state corresponds to a period of data collection between 1 and 2 minutes after administration of the inducing anesthetics. The exact time of the “induction” state varied depending on the patient's response to include a window of time around the loss of eyelash reflex to gentle prodding, which is defined herein as the moment of loss of consciousness. The “1.0 MAC” and “steady MAC” states correspond to the maintenance phase of anesthesia achieved by the maintenance anesthetic, sevofluorane. The “1.0 MAC” state represents a transitional state towards a steady state, and the “steady MAC” state represents an anesthetic state that is deep enough for surgical stimuli. The “emergence” state occurs within the “emergence” period, and represents a transitional state of awakening with some responsiveness to commands but not full consciousness, and includes a window of time around spontaneous opening of the eyes.

Differential EEG signals were obtained by calculating differences between digital EEG signals acquired from neighboring electrodes. Various frequency ranges were analyzed according to their ability to discriminate states by power. The frequency ranges analyzed were 0 to 4 Hz, 4 to 8 Hz, 8 to 12 Hz, 12 to 16 Hz, 16 to 20 Hz, 20 to 30 Hz, 30 to 50 Hz and 50 to 80 Hz.

EEG signals showed distinct high-frequency complex activity during the “baseline” period, and then transitioned to lower-frequency, more rhythmic activity during the “administration” period. Time-frequency analysis using the wavelet transform revealed that the power of EEG signals at frequencies above 20 Hz was high during the “baseline” state, and then significantly diminished with administration of anesthesia, whereas EEG signals in the frequency range below 20 Hz exhibited an opposite trend.

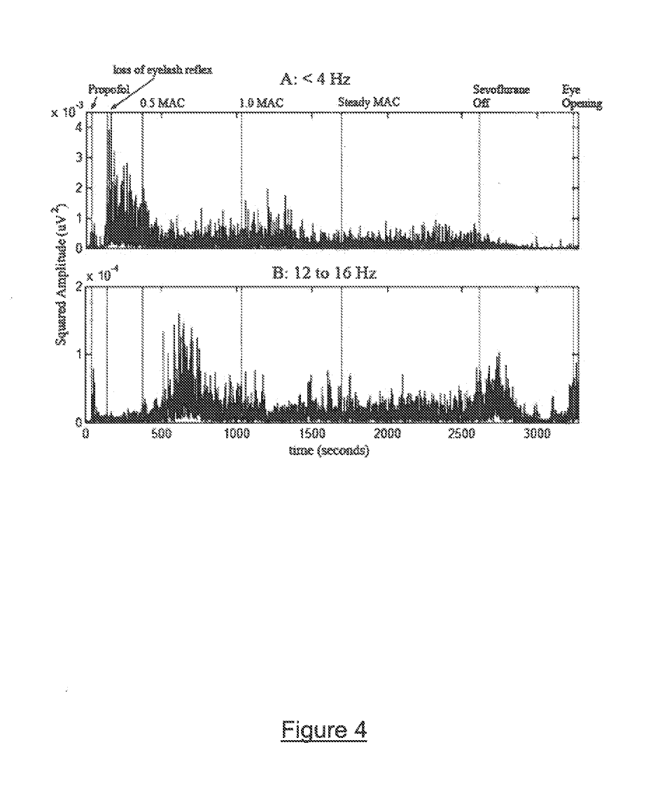

To quantify these changes, normalized power was calculated as described above and with reference to Equations (1) and (2). Differential EEG signals were decomposed into narrow band-limited signals using a finite impulse response (FIR) filter of order 5000. FIG. 4 shows graphical plots of squared amplitude band-limited signals. For each patient, normalized power was calculated for each of the frequency ranges, at each of the five anesthetic states, for the frontal region (F3-Fz and F4-Fz electrode pairs) and the posterior region (P3-Pz and P4-Pz electrode pairs). The average normalized power in the frontal and posterior regions of the six (6) patients during the “baseline” state is summarized in Table 1 below for each frequency range.

TABLE 1

| | |
|---|---|
| Frontal | Posterior |

| | Average | Standard | Average | Standard |
|---|---|---|---|---|
| Frequency | (uV²) | Deviation | (uV²) | Deviation |
| 0 to 4 Hz | 180.44 | 66.26 | 14.76 | 6.21 |
| 4 to 8 Hz | 12.15 | 4.34 | 3.37 | 0.42 |
| 8 to 12 Hz | 8.87 | 2.31 | 4.54 | 0.91 |
| 12 to 16 Hz | 8.05 | 2.14 | 4.26 | 1.71 |
| 16 to 20 Hz | 7.38 | 2.30 | 3.58 | 1.49 |
| 20 to 30 Hz | 23.99 | 8.74 | 8.46 | 4.00 |
| 30 to 50 Hz | 62.08 | 26.65 | 16.97 | 9.72 |
| 50 to 100 Hz | 182.28 | 103.67 | 40.13 | 24.72 |

During the “baseline” state, the average normalized power in the low frequency range (<4 Hz) was 18.89±6.49 times higher in the frontal region than in the posterior region. This large discrepancy was observed to decrease quickly upon introduction of the induction anesthetics, which reduced the average normalized power in the frontal region by 12.60±1.28 fold within seconds of application (not shown). In contrast, the average normalized power in the posterior region was much less affected by the introduction of the induction anesthetics, and decreased by only 1.78±0.41 fold (not shown). Power remained low in both regions for a brief time period, ranging from 50 to 250 seconds depending on the reaction time of each patient to the anesthetics. The average normalized power then suddenly increased and reached a maximum in 8.17±3.48 seconds after the loss of eyelash reflex, as shown in FIG. 4A. After reaching this maximum, the average normalized power then gradually decreased in both regions until stabilizing at a level comparable to that in the frontal region during the baseline state (152.0±45.8% , p=0.343) but was 6.79±1.02 times higher than that in the posterior region during the baseline state (p=0.00001). Overall, at the low frequency range (<4 Hz), the high power observed in the frontal region was reduced by the anesthetics, and decreased to below the average normalized power in the posterior region during the “administration” period.

Average normalized power in the 4 to 8 Hz frequency range followed a similar trend to that of the <4 Hz frequency range, but was more sensitive to the maintenance anesthetic, sevoflurane. After a reduction of power during the “induction” state, the average normalized power in this range was observed to increase monotonically from the start of sevoflurane until reaching the “steady MAC” state. The average normalized power during the “steady MAC” state was 73.0% (p=0.047) and 109.9% (p=0.0091) greater than that during the “1.0 MAC” state for the frontal and posterior regions, respectively. The average normalized power decreased during the “emergence” state and remained low for several minutes following the cessation of sevoflurane, but increased to that of the “baseline” state or greater as patients regained consciousness. The average normalized power in the 4 to 8 Hz frequency range was stronger in the frontal region during the awake state, but to lesser degree than was observed in the <4 Hz frequency range, as may be seen in Table 1.

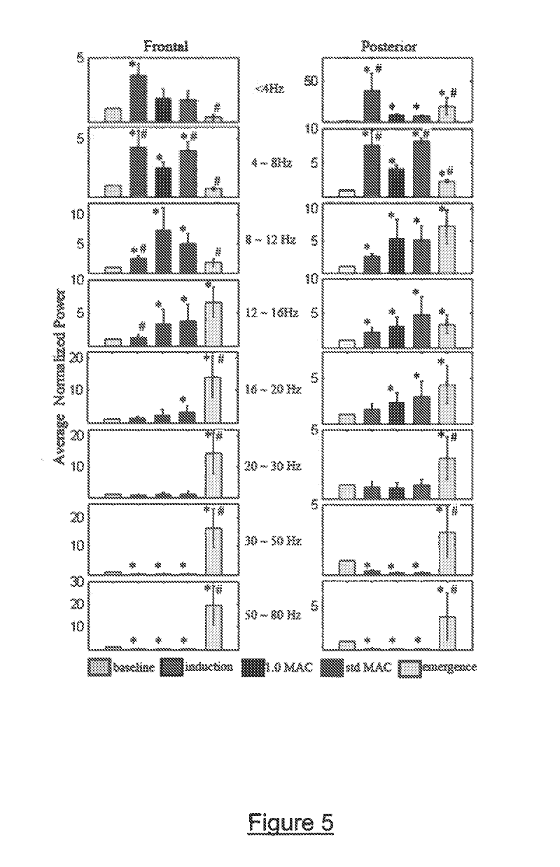

Average normalized power in the 8 to 12 Hz frequency range and the 12 to 16 Hz frequency range was initially very small during the “baseline” state, and then increased considerably upon administration of sevoflurane following introduction of propofol/fentanyl, as may be seen in FIG. 5. The average normalized power in the 12 to 16 Hz frequency range was particularly sensitive to sevoflurane, as both the starting and stopping of sevoflurane resulted in abrupt increases in average power, as may be seen in FIG. 4B. This increase began several minutes after the start of sevoflurane, and peaked subsequent to reaching 0.5 MAC but several minutes before reaching the “1.0 MAC” state. The average normalized power then decreased, and remained at a steady value until sevoflurane was turned off. At the termination of sevoflurane, the average normalized power was observed to increase abruptly, before returning to the level of the “baseline” state, as may be seen in FIG. 4B. This trend was observed in both the frontal and posterior regions.

Average normalized power in the 16 to 20 Hz frequency range increased slightly during administration of anesthesia, while normalized power in the 20 to 30 Hz frequency range did not show significant change during administration of anesthesia. However, during patient recovery in the “emergence” state, average normalized power in the >30 Hz frequency range increased greatly in both the frontal and posterior regions, with larger increases in the frontal region. Additionally, the average normalized power in the >30 Hz frequency range was high during the “baseline” state, but abruptly decreased during the “induction” state and remained low during the “administration” period until recovery during the “emergence” state.

Communication between neuronal ensembles lies at the heart of information coding and binding in the brain, as discussed in the publications by M. Joliot, U. Ribary and R. Llinas, entitled “Human Oscillatory Brain Activity near 40 Hz Coexists with Cognitive Temporal Binding,” PNAS, vol. 91, pp. 11748-11751, 1994, and by G. Buzsaki and A. Draguhn, entitled “Neuronal Oscillations in Cortical Networks,” Science, vol. 304, pp. 1926-1929, 2004. Such communication can be quantified by measuring the phase-to-phase coupling between signals obtained from different brain regions. Relative phase coherence, which quantifies the relative degree of phase coordination between two signals, provides a good indication of the phase-to-phase coupling. Relative phase coherence was calculated from the continuous wavelet transform as described above and with reference to Equation (6), for various electrode pairs, including bilateral electrode pairs (F7-F3 and F8-F4), (T3-C3 and T4-C4) and (T5-P3 and T6-P4), as well as anterior-posterior pairs (F8-F4 and T4-C4), (F8-F4 and T6-P4), (T4-C4 and T6-P4), (F7-F3 and T3-C3), (F7-F3 and T5-P3) and (T3-C3 and T5-P3).

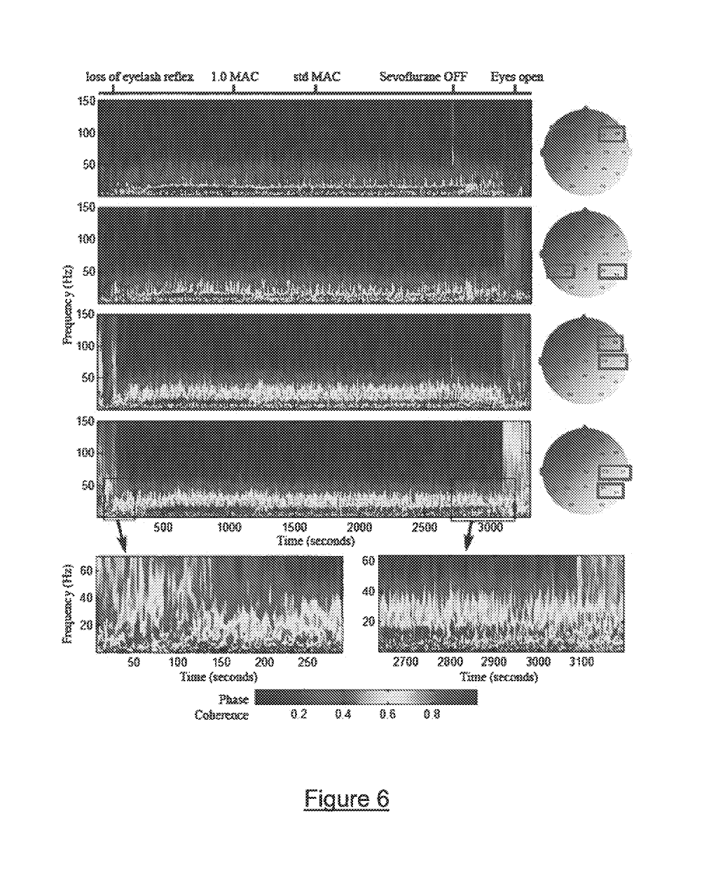

Exemplary phase coherence plots of these electrode pairs are shown in FIG. 6, in which the upper two graphical plots show the relative phase coherence of bilateral electrode pairs, and the lower two graphs show the phase coherence of right-hemisphere anterior-posterior electrode pairs.

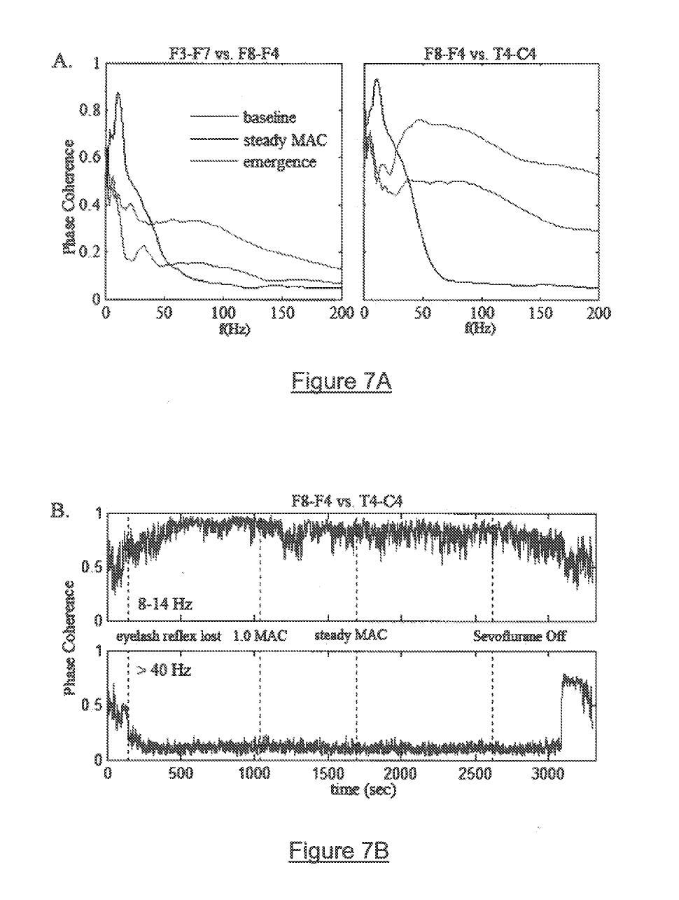

During the “baseline” state, strong relative phase coherence was observed at frequencies below 15 Hz for all electrode pairs, with a peak in the 5 to 9 Hz frequency range, as may be seen in FIGS. 6 and 7A. The anterior-posterior pairs also showed significant relative phase coherence (>0.4) at frequencies above 30 Hz during the “baseline” state (see FIG. 7A). During the induction of anesthesia, frequency ranges having significant relative phase coherence (>0.4) became broader and, by the time of loss of eyelash reflex, relative phase coherence at frequencies below 40 Hz was significant. Meanwhile, relative phase coherence for frequencies above 50 Hz remained very low during administration of anesthesia following the loss of eyelash reflex, despite some signals from some electrode pairs exhibiting high relative phase coherence at frequencies above 50 Hz during the baseline period.

Significant relative phase coherence was observed for all electrode pairs during the maintenance phase in the 8 to 14 Hz frequency range (i.e. the “alpha rhythm” frequency range), as may be seen in FIGS. 6 and 7A. The average relative phase coherence in the 8 to 14 Hz frequency range before anesthesia was 0.369±0.034 (0.326±0.093 and 0.390±0.087 in bilateral and anterior-posterior electrode pairs, respectively), which then increased to 0.678±0.052 (0.619±0.044 and 0.708±0.088, in bilateral electrode pairs and anterior-posterior electrode pairs, respectively) during the “steady MAC” state. It is important to note that the relative phase coherence of alpha rhythm between bilateral electrode pairs was higher in the posterior region (0.435±0.045) than in the frontal region (0.220±0.031). However, this was reversed during the “steady MAC” state, when the relative phase coherence of the posterior region (0.621±0.062) was slightly lower than that of the frontal region (0.661±0.078). Several minutes after termination of sevoflurane, this strong relative phase coherence in the alpha rhythm began to decrease, followed by a sudden increase of relative phase coherence at frequencies above 40 Hz, as may be seen in FIG. 7B. This increase in coherence at frequencies above 40 Hz was most prominent in the anterior-posterior electrode pairs, which were the same electrodes that also showed significant relative phase coherence at frequencies above 40 Hz during the “baseline” state, as may be seen in FIGS. 6 and 7A. Even for the bilateral electrode pairs, where high-frequency relative phase coherence was absent during baseline, a noticeable increase in high-frequency phase coherence was observed, especially for the posterior bilateral pairs (T5-P3 and T6-P4). As patients began regaining consciousness, this high-frequency relative phase coherence subsided. It is also interesting to note that the strong high-frequency relative phase coherence was briefly seen during induction a few seconds before the loss of eyelash reflex, and before the appearance of the strong relative phase coherence in the alpha rhythm, as may be seen in FIG. 6, inset.

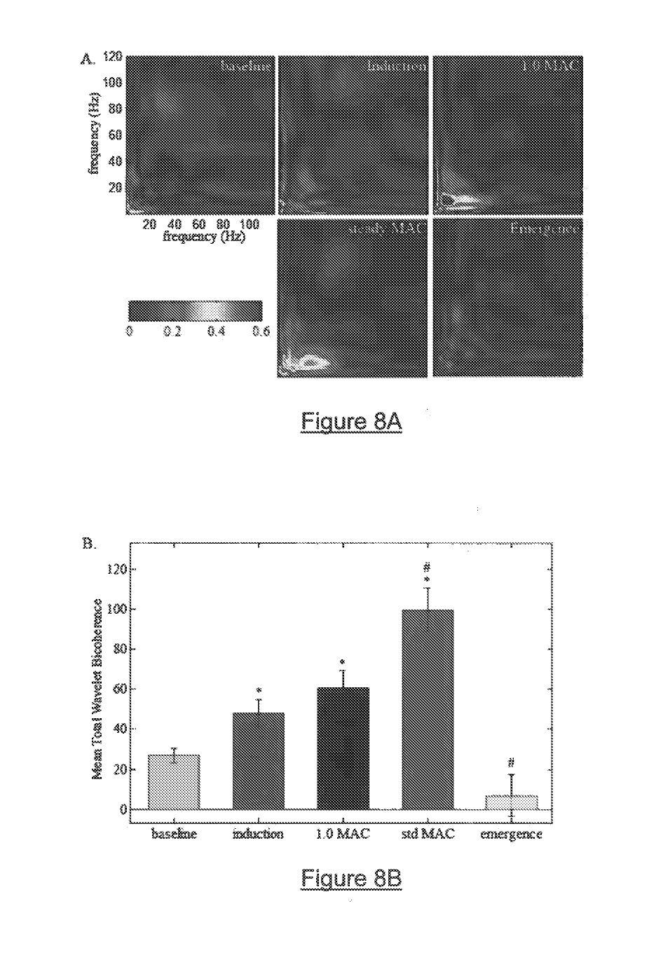

Wavelet bicoherence can be used to quantify second-order interactions occurring between signals during cross-frequency coupling. The wavelet bicoherence was calculated as described above and with reference to Equation (8), for signals obtained from bilateral frontal electrodes (F7-F3 vs. F8-F4), bilateral central electrodes (T3-C3 vs. T4-C4), ipsilateral frontal to central electrodes (F8-F4 vs. T4-C4), and ipsilateral central to posterior electrodes (T4-C4 vs. T6-P4). Strong wavelet bicoherence was observed in the 5 to 18 Hz and 10 to 40 Hz frequency ranges during anesthesia. This phenomenon was most readily seen between bilateral and frontal electrodes. As with relative phase coherence, because wavelet bicoherence is an averaged quantity over a period of time, the choice of window size can influence the measured wavelet bicoherence. To this end, window sizes of 1 to 5 seconds, using 1 second increments and a 50% window overlap, were investigated. A 5 second window size was used in this Example, as it produced the most consistent and prominent results, particularly in the 5 to 18 Hz and 10 to 40 Hz frequency ranges. FIG. 8A shows exemplary wavelet bicoherence plots of one patient during the anesthesia protocol, with each frame representing one of the five anesthetic states. Strong wavelet bicoherence in the 5 to 18 Hz and 10 to 40 Hz frequency ranges became evident as the patient entered the maintenance phase, namely during the “1.0 MAC” and “steady MAC” states. An image clustering technique using an 8-connected neighbor search was utilized to define a bi-frequency region of interest. The total strength of wavelet in these identified clusters per frame was calculated to quantify the strength of wavelet in the 5 to 18 Hz and 10 to 40 Hz frequency ranges. The average wavelet bicoherence over 30 second windows around each of the five anesthetic states was computed for all patients, and is graphically plotted in FIG. 8B. As may be seen, the wavelet bicoherence significantly increased upon introduction of the induction anesthetics, and then further increased upon the introduction of the maintenance anesthetics, but decreased during emergence.

In addition to phase and cross-frequency coupling, neuronal systems communicate through phase-to-amplitude coupling, as described in the above-referenced publications by Canolty et al. and by Buzaki et al., and in the publications by W. D. Penny, E. Duzel, K. J. Miller and J. G. Ojemann entitled “Testing for nested oscillation,” Journal of Neuroscience Methods, vol. 174, pp. 50-61, 2008, and by J. Lisman entitled “The theta/gamma discrete phase code occurring during the hippocampal phase precession may be a more general brain coding scheme,” Hippocampus, vol. 15, pp. 913-922, 2005. The amplitude of neuronal activity at one frequency range can be coordinated with the phase of activity at another, typically lower frequency range. The modulation index measures this phase-to-amplitude coupling by computing a correlation between the amplitude of one frequency and the phase of another frequency. Modulation index was calculated as described above and with reference to Equation (12) for the following electrode pairs: F7-F3, T3-C3, T5-P3, F8-F4, T4-C4 and T6-P4. The frequency range used for the amplitude signal was 0 to 200 Hz with 5 Hz increments, while the frequency range used for the phase signal was 0 to 30 Hz with 1 Hz increments. Each modulation index was calculated using 6 seconds of EEG signal duration, with a 3 second overlap.

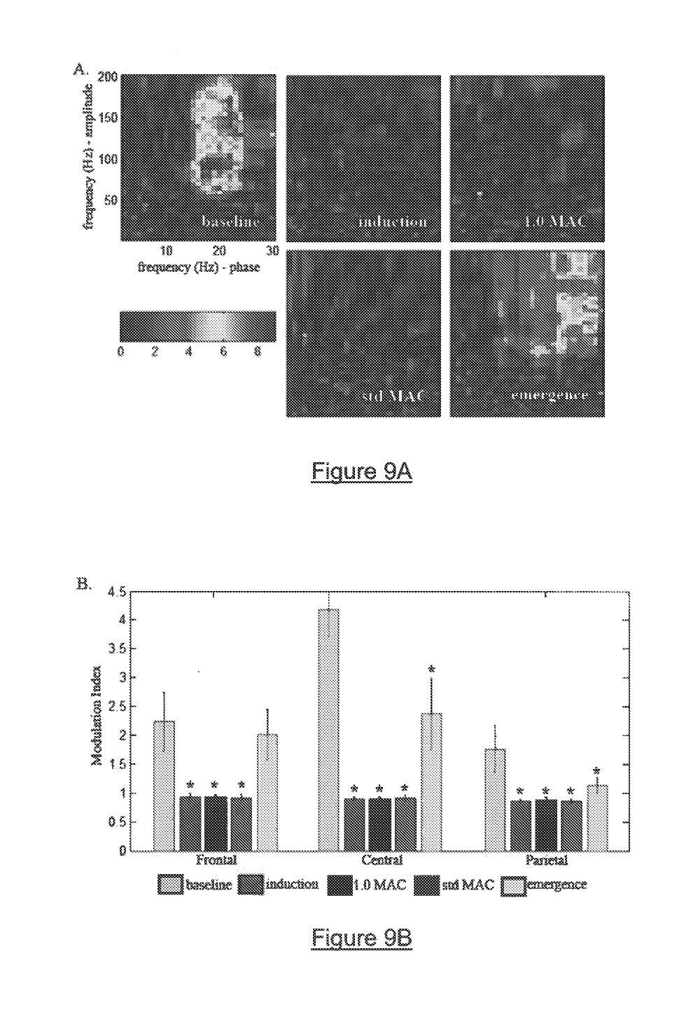

As may be seen in FIG. 9A, a strong modulation index between the frequency ranges of 15 to 30 Hz (phase) and 60 to 200 Hz (amplitude) was observed in awake patients for all electrode pairings, suggesting strong phase-to-amplitude coupling between beta and gamma activities (“beta-gamma coupling”). The value of modulation index was initially high during the induction phase, but began decreasing several seconds before the loss of eyelash reflex. The beta-gamma coupling was longest lived in the temporal-central regions, persisting for 11.7 and 12.3 seconds longer than that of the frontal and temporal-parietal regions, respectively (not shown). In addition, the beta-gamma coupling was also strongest in the temporal-central region with a modulation index of 4.16±1.15, as compared with 2.22±1.23 for the frontal region and 1.75±0.99 for the temporal-parietal region, as measured during the baseline period. These results suggest that beta-gamma coupling is most prominent in the temporal-central region of the brain during the awake state.

The value of the modulation index significantly decreased in all three regions of the brain several seconds after the introduction of induction agents, and even prior to the loss of eyelash reflex. The value of modulation index then remained below 1 throughout the entire “administration” period. This result suggests that the communication between and within the neuronal systems is disrupted by anesthetics. Although a full recovery from general anesthesia typically takes hours, patients regain consciousness within minutes after termination of anesthetics, the first sign of regained consciousness being spontaneous opening of the eyes. Average modulation indices of the six (6) patients calculated at the time of spontaneous eye opening showed partial recovery, with values of 1.99±0.44, 2.35±0.62 and 1.12±0.13 for frontal, temporal-central, and temporal-parietal regions, respectively. The average modulation indices of the six (6) patients for each of the three regions considered are summarized in FIG. 9B. Overall, the modulation index showed a high sensitivity to the transition during the early induction phase of anesthesia.

Without the ability to communicate with patients under anesthesia, the precise anesthetic state of the patient is difficult to ascertain. This is especially problematic since patients typically reach a state of unconsciousness within minutes of the administration of induction agents and remain unconscious after switching to a maintenance anesthetic such as sevoflurane, without additional outwardly-observable physiological signs. Although loss of consciousness is typically defined as a loss of response to mild prodding, as described in the publication by J. W. Johansen, entitled “Update on bispectral index monitoring,” Best Practice Clinical Anaesthesiology, vol. 20, no. 1, pp. 81-99, 2006, after the patient reaches loss of consciousness, the patient experiences continuously changing levels of consciousness despite the absence of obvious physiological signs. It is more than likely that these changes in consciousness correspond with changes in the brain's electrical activity.

However, several signal features observed during this study correlated more strongly with the initial loss of consciousness, and namely the loss of eyelash reflex. These signal features include an increase of low frequency (<4 Hz) power, an abrupt decrease of high-frequency (>25 Hz) power, and an abrupt decrease in the modulation index of beta and gamma rhythms. Such an increase of low frequency power and a decrease of high-frequency power have been described in association with state of sedation and with light anesthesia in previous studies, namely in the publications by A. Cimenser et al., entitled “Tracking brain states under general anesthesia by using global coherence analysis,” PNAS, vol. 108, no. 21, pp. 8832-8837, 2011, by B. Antkowiak and H. Hentschke, entitled “Cellular mechanisms of gamma rhythms in rat neocortical brain slices probed by the volatile anaesthetic isoflurane,” Neuroscience Letters, vol. 231, pp. 87-90, 1997, and by A. Nayak, R. J. Roy and A. Sharma, entitled “Time-frequency spectral representation of the EEG as an aid in the detection of depth of anesthesia,” Annals of Biomedical Engineering, vol. 22, pp. 501-513, 1994.

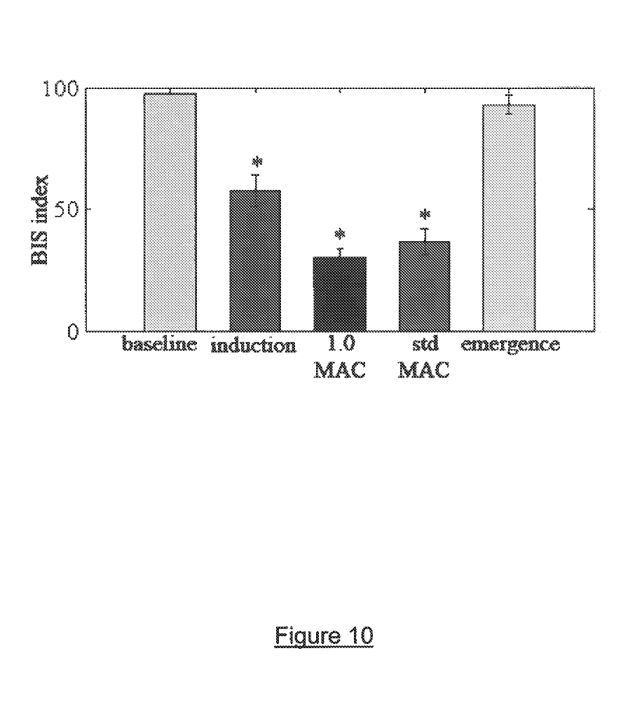

Additionally, concurrently recorded Bispectral Index (BIS™) showed sensitivity at detecting the loss of consciousness, with the largest drop in the Bispectral Index occurring around the time of loss of eyelash reflex, as may be seen in FIG. 10. Although an additional significant decrease in the Bispectral Index was observed between the “induction” and “1.0 MAC” states, the majority of this decrease occurred prior to administration of the maintenance anesthetic, sevoflurane. The average Bispectral Index several seconds before the start of sevoflurane was 37.42±7.19 (not shown), which is not significantly different from the average Bispectral Index during the “1.0 MAC” state, which was 33.44±10.11 (p>0.05). These results suggest that the main difficulty inherent in monitoring DoA is identifying and measuring changes in the levels of consciousness following the event of loss of conciousness, especially during the maintenance phase of anesthesia.

However, it has been observed during this study that some signal features are sensitive to change during the maintenance phase of anesthesia. One such feature is the wavelet bicoherence between the 5 to 18 Hz and 10 to 40 Hz frequency ranges, which showed a monotonically increasing trend during the maintenance phase until the “steady MAC” state was reached, as shown in FIG. 8B. Additionally, the normalized power in the 4 to 8 Hz frequency range also showed a monotonically increasing trend during the maintenance phase of the “administration” period until reaching the “steady MAC” state, especially in the posterior region, as shown in FIG. 5 for the case of average normalized power.

Although in the above embodiments, the pre-processing module, the processing module, the display and the memory are described as being embodied within a single general purpose computing device, in other embodiments, the pre-processing module, the processing module, the display and the memory may alternatively form part of another device, such as for example a medical instrument or other medical equipment. Still alternatively, the pre-processing module, the processing module, the display and the memory may be embodied in two or more devices.

Although in embodiments described above, differential EEG signals are obtained by calculating differences between digital EEG signals, each of which is obtained by amplifying a respective analog EEG signal as necessary, and converting the amplified analog EEG signals to digital, in other embodiments, the differential EEG signals may alternatively be obtained in other ways. For example, in one alternative embodiment, differences may be calculated between analog EEG signals, amplified as necessary, and then converted to digital to obtain the differential EEG signals.

Although embodiments have been described above with reference to the accompanying drawings, those of skill in the art will appreciate that variations and modifications may be made without departing from the scope thereof as defined by the appended claims.

What is claimed is:

1. A method of monitoring depth of anesthesia comprising:
generating differential EEG signals from EEG signals acquired by electrode pairs during an anesthesia protocol;
processing said differential EEG signals to determine at least one signal feature; and
monitoring changes in said at least one signal feature to determine depth of anesthesia, wherein said at least one signal feature comprises at least one of normalized power of said differential EEG signals acquired from at least one of said electrode pairs, and wavelet bicoherence between said differential EEG signals acquired from at least two of said electrode pairs.

2. The method of claim 1, wherein the wavelet bicoherence is calculated between 5 to 18 Hz and 10 to 40 Hz frequency ranges of said differential EEG signals.

3. The method of claim 1, wherein the normalized power is calculated within a 4 to 8 Hz frequency range of said differential EEG signals.

4. The method of claim 1, further comprising:
calculating a required dosage value of anesthesia based on said monitoring.

5. The method of claim 4, further comprising delivering a controllable quantity of anesthesia to a patient based on said calculated required dosage value.

6. The method of claim 1, further comprising delivering a controllable quantity of anesthesia to a patient based on a calculated required dosage value.

7. The method of claim 2, wherein the normalized power is calculated within a 4 to 8 Hz frequency range of said differential EEG signals.

8. The method of claim 7, further comprising:
calculating a required dosage value of anesthesia based on said monitoring.

9. The method of claim 8, further comprising delivering a controllable quantity of anesthesia to a patient based on said calculated required dosage value.

10. A system for monitoring depth of anesthesia comprising:
electrodes configured to be placed on subject's scalp; and
processing structure for generating differential EEG signals from EEG signals acquired by electrode pairs during an anesthesia protocol, said processing structure being configured to:
process said differential EEG signals to determine at least one signal feature; and
determine changes in said at least one signal feature during said anesthesia protocol, wherein said at least one signal feature comprises at least one of normalized power of said differential EEG signals acquired from at least one of said electrode pairs, and wavelet bicoherence between said differential EEG signals acquired from at least two of said electrode pairs.

11. The system of claim 10, wherein the wavelet bicoherence is calculated between 5 to 18 Hz and 10 to 40 Hz frequency ranges of said differential EEG signals.

12. The system of claim 10, wherein the normalized power is calculated within a 4 to 8 Hz frequency range of said differential EEG signals.

13. The system of claim 10, wherein the processing structure is configured to automatically calculate a required dosage value of anesthesia based on said changes in said at least one signal feature.

14. The system of claim 13, further comprising dosage administration structure configured to deliver a controllable quantity of anesthesia to a patient via a fluid delivery line.

15. The system of claim 14, wherein said dosage administration structure is further configured to deliver said controllable quantity of anesthesia based on said calculated required dosage value.

16. The system of claim 11, wherein the normalized power is calculated within a 4 to 8 Hz frequency range of said differential EEG signals.

17. The system of claim 16, wherein the processing structure is configured to automatically calculate a required dosage value of anesthesia based on said changes in said at least one signal feature.

18. The system of claim 17, further comprising dosage administration structure configured to deliver a controllable quantity of anesthesia to a patient via a fluid delivery line.

19. The system of claim 18, wherein said dosage administration structure is further configured to deliver said controllable quantity of anesthesia based on said calculated required dosage value.

20. A non-transitory computer-readable medium having embodied thereon a computer program for monitoring depth of anesthesia, said computer program comprising instructions which, when executed by processing structure, cause an apparatus at least to:
process differential EEG signals generated from EEG signals acquired by electrode pairs during an anesthesia protocol to determine at least one signal feature; and
monitor changes in said at least one signal feature to determine depth of anesthesia, wherein said at least one signal feature comprises at least one of normalized power of said differential EEG signals acquired from at least one of said electrode pairs, and wavelet bicoherence between said differential EEG signals acquired from at least two of said electrode pairs.

21. An apparatus comprising:
processing structure; and
memory in communication with the processing structure, the memory storing computer-readable code, the computer-readable code when executed by the processing structure causing the apparatus at least to:
process differential EEG signals generated from EEG signals acquired by electrode pairs during an anesthesia protocol to determine at least one signal feature; and
monitor changes in said at least one signal feature to determine depth of anesthesia, wherein said at least one signal feature comprises at least one of normalized power of said differential EEG signals acquired from at least one of said electrode pairs, and wavelet bicoherence between said differential EEG signals acquired from at least two of said electrode pairs.

22. The apparatus of claim 21, wherein the wavelet bicoherence is calculated between 5 to 18 Hz and 10 to 40 Hz frequency ranges of said differential EEG signals.

23. The apparatus of claim 21, wherein the normalized power is calculated within a 4 to 8 Hz frequency range of said differential EEG signals.

24. The apparatus of claim 22, wherein the normalized power is calculated within a 4 to 8 Hz frequency range of said differential EEG signals.
